(19) United States
(12) Patent Application Publication
(10) Pub. No.: US 2025/0176903 A1
(43) Pub. Date: Jun. 5, 2025

(54) Wearable Energy Stimulation System For Mammals Targeting The Vagus Nerve, Includes Electrical And Haptic Energy Emitters, A Collar Coupling Apparatus, Energy-Emitter Positioning Circuits, And Position Maintenance And Verification Means, Multi-Modal Operation, Energy-Emitter Modularization, And Closed Loop Configuration

(71) Applicants: Jonathan M. Honeycutt, El Paso, TX (US); Thomas A. La Rovere, Newport, WA (US)

(72) Inventors: Jonathan M. Honeycutt, El Paso, TX (US); Thomas A. La Rovere, Newport, WA (US)

(21) Appl. No.: 17/874,080

(22) Filed: Jul. 26, 2022

(51) Int. Cl. A61B 5/00; A61B 5/0205; A61B 5/021; A61B 5/024; A61B 5/08; A61B 5/11; A61B 5/145; A61H 23/00; A61H 23/02; A61N 1/02; A61N 1/04; A61N 1/36; G16H 40/67

(52) U.S. Cl. CPC A61B 5/4836; A61B 5/0205; A61B 5/021; A61B 5/02405; A61B 5/02416; A61B 5/02438; A61B 5/0816; A61B 5/1118; A61B 5/14542; A61B 5/4035; A61B 5/6802; A61B 5/7257; A61B 5/7282; A61B 5/7405; A61B 5/742; A61H 23/006; A61H 23/0218; A61N 1/025; A61N 1/0456; A61N 1/0484; A61N 1/36034; G16H 40/67; A61B 2560/0214; A61B 2560/0462; A61B 2562/0219; A61B 2562/0223; A61H 2201/10; A61H 2201/1611; A61H 2201/165; A61H 2201/501; A61H 2201/5043; A61H 2201/5058; A61H 2201/5097; A61H 2205/04

(57) ABSTRACT

A vagus nerve stimulation system for mammals includes signal generating, conditioning, and control electronics, stimulation monitoring electronics, and a collar coupling apparatus configured for coupling energy emitters anatomically proximal to the cervical branch of the vagus nerve. Collar coupling apparatus includes multiple, position-stabilizing anti-rotation bodies, at least one position verification indicator, and a movable car housing energy emitter circuits or an inductive energy transducer. Two energy modalities are available via electrodes for electrical stimulation and induction-powered haptic emitters for vibrational stimulation. In an advanced configuration, a stimulation transducer produces inductive energy transmitted to an energy receiver-emitter package percutaneously introduced in the subcutaneous layer proximal to the vagus nerve target. Subcutaneous energy receiver-emitters include electrical and haptic variants, both having magnetic compositions which enable spatial position matching with external transducers using Hall sensor technology. In the haptic receiver-emitter variant, a magnetic composition produces haptic stimulation via electromagnetic induction from the external transducer. A further configuration includes an RFID tag in the subcutaneously deposited energy receiver-emitter package. A modularized configuration offers selectable, interchangeable energy emitter packages for electric and haptic stimulation. Stimulation control electronics are comprised within an external computerized device having a GUI for user parameterization of stimulation. A closed loop stimulation variant operates according to algorithmic programming incorporating cardiorespiratory sensor feedback and divisional monitoring of autonomic nervous system indicia and sensor actigraphy.

### CROSS-REFERENCE TO RELATED APPLICATIONS

This application claims priority of Provisional Application No. 63/225,780 filed on Jul. 26, 2021 by inventors Jonathan M. Honeycutt and Thomas A. La Rovere.

### FIELDS OF INVENTION

The present invention includes apparatus and methods belongs to the emerging fields of neurostimulation and neurostimulation-enhanced interventional therapeutics for health, healing and wellness. Neurostimulation may be broadly defined as the application of energy to nerves targeted directly or indirectly (e.g., by applying energy to surrounding, connecting and/or conductive tissues, e.g., skin, tissues and vasculature), for the purpose of producing beneficial changes in the activity of neurotransmitters, the activity within structures and centers of the brain, in neuronal and synaptic activity, and as streams of cascading effects producing changes in the activity of organs, particularly organs in communication with the autonomic nervous system. The present invention includes multiple forms of energy stimulation or neurostinulation, including electrical and haptic (or vibrational) energy,

Hereinafter, the terms “neuromodulation,” “neurostimulation,” “neurotherapy” and “nerve stimulation” are used interchangeably and refer to the full range of therapeutic modalities and methods by which energy, as electricity, electromagnetic, and haptic energy, may be electronically delivered to anatomical structures of a mammalian body, such structures including nerves, tissues including connective tissues, organs individually and systemically, muscles, vasculature, glands; and to refer to devices designed and used to accomplish such energy delivery through direct or indirect application to said anatomic structures, using external, non-invasive, transcutaneous means and through the use of subcutaneously implanted energy emitters.

### BACKGROUND OF THE INVENTION

The present disclosure relates to a system of apparatus and methods for neurostimulation applied to mammals. Research in the field of transdermal neural stimulation has provided insights about the neurological pathways available through energy stimulation of the vagus nerve in mammals. In many mammals, such as domesticated dogs and cats, the vagus nerve and its functional neurological activity essentially mirrors its anatomy and neurological functions in human beings. The health benefits of vagus nerve stimulation in human beings are well established, with a growing list of clinical, health and wellness applications developed over three decades of research now comprising over 20,000 research projects. Pre-clinical research in vagus nerve stimulation has often used non-human mammals, such as laboratory mice and canines, and employed implanted stimulation devices or external devices wired through the skin and connected to the vagus nerve via invasive neurosurgery. Due to the cost and the risks of surgical implantation, neither of these device-nerve coupling schemes is practical for use with domesticated animals such as canines, felines, horses and the like.

Transdermal (also called “transcutaneous”) stimulation of the vagus nerve using electrodes in contact with visible skin-surface landmarks (e.g., in the auricular nerve field) is now a proven method of neurological therapy in human beings. Realizing the benefits of transdermal vagus nerve stimulation in mammals, however, presents several challenges, starting with the fact that, unlike human beings, animals will not be voluntary recipients of vagus nerve stimulation, will not participate in the set-up or administration of vagus nerve stimulation, will not understand the sensation produce by stimulation and may engage in aversive behavioral such as avoidance, active resistance and escape. These and other factors may prevent the use of electrodes attached to the auricular nerve field which could be disturbed or even dislodged by scratching and pawing and other potentially disruptive behavior.

### Object and Advantages

Research in the field of transdermal neural stimulation has provided insights about the neurological pathways available through energy stimulation of the vagus nerve in mammals. In many mammals, such as domesticated dogs and cats, the vagus nerve and its functional neurological activity essentially mirrors its neurological functions in human beings. The health benefits of vagus nerve stimulation in human beings are well established, with a growing list of clinical, health and wellness applications developed over three decades of research now comprising over 30,000 research papers. Pre-clinical research in vagus nerve stimulation has often used non-human mammals, such as laboratory mice, felines and canines, and employed implanted stimulation devices or external devices wired through the skin and connected to the vagus nerve via invasive neurosurgery. Due to the cost and the risks of surgical implantation, neither of these device-nerve coupling schemes is practical for use with domesticated animals such as canines, felines, horses and the like.

Transdermal (also called “transcutaneous”) stimulation of the vagus nerve using electrodes in contact with visible skin-surface landmarks (e.g., in the auricular nerve field) is now a proven method of neurological therapy in human beings. Realizing the benefits of transdermal vagus nerve stimulation in mammals, however, presents several challenges, starting with the fact that, unlike human beings, animals will not be voluntary recipients of vagus nerve stimulation, will not participate in the set-up or administration of vagus nerve stimulation, will not understand the sensation produce by stimulation and may engage in aversive behavioral such as avoidance, active resistance and escape. These and other factors may prevent the use of electrodes attached to the auricular nerve field which could be disturbed or even dislodged by scratching and pawing and other potentially disruptive behavior.

### Collar Challenges

The present vagus nerve stimulation system employs a collar-based coupling system configured to resist aversive behavioral responses of many animals, in particular canines, felines, horses, and other domesticated mammals. Mounting energy emitters on a neck-worn collar presents multiple challenges such as the positional-stability of the collar relative to the nerve target and the fur barrier between electrodes and the skin. Maintaining the positional stability of the collar against displacing rotational forces without constricting the animal's neck and producing pain, discomfort and aversive response is a significant and, until now unsolved challenge. Collars must be installed and worn humanely, without choking the animal. Energy emitter configured as electrodes used for electrically stimulating an animal's vagus nerve must make and maintain contact with the animal's skin directly over the nerve target. The problem is, humanely worn collars may rotate around the animal's neck and even slight rotation can displace electrodes relative to the nerve target and thereby prevent or reduce effective stimulation of the nerve target.

### Electrode Challenges

Composing neurostimulation devices for use with mammals presents a number of challenges. Chief among these are the maintenance of electrode-skin contact, and the positioning and stability of electrodes and other energy emitters relative to the nerve target. The cervical branch of the vagus nerve target presents a narrow target profile, typically only 3 to 7 millimeters in width, depending on the size of the animal.

Disclosed in the present invention is a collar-based stimulation device that maintains electrode contact by monitoring resistance and incorporating springs in the electrode package to automatically adjust the electrodes' “ride” and pressure against the skin. Positioning of the electrodes is accomplished by, first, palpating the carotid sheath in which the vagus nerve target enclosed to locate the nerve target; and, secondly by sliding a movable car carrying electrodes to a matching position on the proximal lateral-distal side of the neck. Stabilizing the collar against rotation around the neck is accomplished by shaped rubber traction bodies composed on the inside of the collar, with upper traction bodies biased against downward collar rotation and lower traction bodies biased against upward collar rotation. Once the collar is installed and the electrode car positioned on the collar proximal to the cervical vagus nerve target in the neck, a visible position indicator located at 12 o'clock on the collar's exterior provides an observable marker of position maintenance by its visually observable alignment with the dorsal mid-line of the animal's neck.

### Two Types of Emitters: External & Internal

The present invention discloses two types of stimulation energy emitters: transcutaneous energy emitters, being completely external, located outside of the animal's body and positioned immediately proximal to an internal nerve target; and subcutaneous, comprising a minute energy receiver-transformer-emitter package inserted via percutaneous needle injector of the type used for tagging animals with RFID circuits. An additional composition of the latter, subcutaneous version combines the miniature energy receiver-transformer-emitter package with an RFID tag to obviate the need for multiple insertion procedures and to serve as a therapeutic access-port for vagus nerve stimulation on an as needed basis, present and future.

In a more advanced configuration, two types of energy emitter are selectable: a haptic emitter of vibrational energy and an electrical emitter of electricity. In both types, the energy emitter is instantly inserted subcutaneously in the target zone proximal to the vagus nerve target (similar to a conventional RFID tag) using a percutaneous wide bore needle injector. Both types of energy emitters are configured as inductive receivers powered by an external inductive transducer-transmitter located on the movable car installed on the collar-coupling apparatus. Both haptic and electrical receiver-emitters include magnets. The haptic receiver-emitter receives electronically controlled and conditioned magnetic induction output from the external inductive transducer-transmitter, according to user-selected stimulation parameters that include vibration frequency, waveform, amplitude, and the like. Rapid polarization switching (or shifting) in the magnetic field produced by the external inductive transducer-transmitter causes the magnetic internal receiver-transmitter to vibrate, thereby producing haptic energy stimulation.

The subcutaneously situated electrical energy receiver-emitter also includes a magnet and receives electronically controlled and conditioned electric induction output from an external, inductive transducer-transmitter mounted on the collar-coupling apparatus. For both haptic and electrical energy emitters, a Hall sensor co-located on the car carrying the inductive transducer-transmitter provides positional feedback of transmitter-receiver alignment.

via light and/or sound emitters, during collar-car installation and for ongoing position monitoring.

### Advantages

Using a collar-based stimulation device avoids the need for expensive neurosurgical implantation of a battery-powered stimulator and the potential costs and complications thereof, including the need to replace expended batteries, the dissection of the nerve and associated wiring to a stimulator, potential nerve damage, and wound healing risks, such as infection.

Included magnetics and Hall sensor technology, combined with the coupling collar's position-verification and position-stabilization (anti-rotation) elements provide an optimal, multi-element solution for maintaining the crucial alignment of emitter-receiver and induction transmitter. Functionally, said transmitter-receiver alignment via Hall sensor can be configured for automatic operation immediately prior to each stimulation session.

### Modularity

The present disclosure includes a modularized neurostimulation system for generating and delivering energy stimulation to the aforesaid anatomic structures of mammals, with particularity to nerves and nerve tissues, muscles and muscle tissues, vasculature and vascular tissues, and skin and skin tissues, to produce a wide variety of therapeutic effects and health benefits, including such effects and benefits as may be realized according to the anatomic location, biological operation, and systemic functionality of one or more selected stimulation targets.

Although numerous studies support the efficacy of vagus nerve stimulation for a variety of clinical indications, further research is needed to established clinical paradigms and protocols for stimulation parameters such as duration and frequency of each stimulation session, length of treatment, electrical frequencies to be used, pulse-widths, waveforms, and for nerve targeting and electrode placements. The modularity of energy emitters enables users, including professional users like veterinarians, to select the type of energy and energy-emitter package they believe most tolerable and suitable for an animal patient or recipient.

### BRIEF DESCRIPTIONS OF THE DRAWINGS

FIG. 1. Illustrates the application of a neck worn collar incorporating an enclosure car housing stimulation control electronics and emitter contacts.

FIG. 2. Illustrates an adjustable means of positioning said enclosure car.

FIGS. 3A-3B. Illustrates various functional embodiments of the systems.

FIG. 3A indicates the functions contained in a mammal worn collar.

FIG. 3B indicates the functions contained in an implanted capsule.

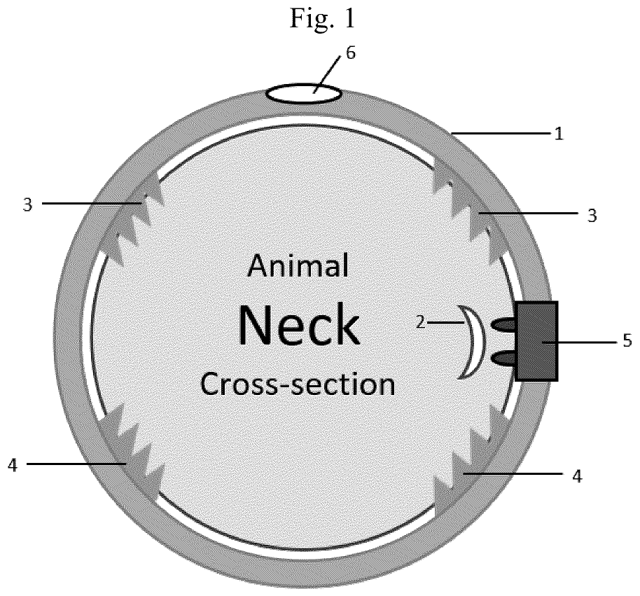

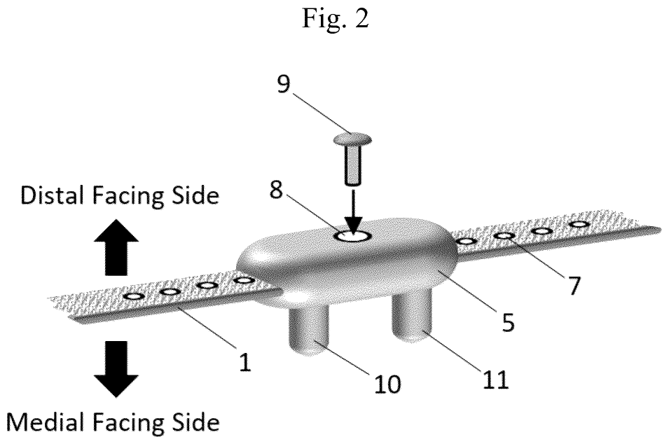

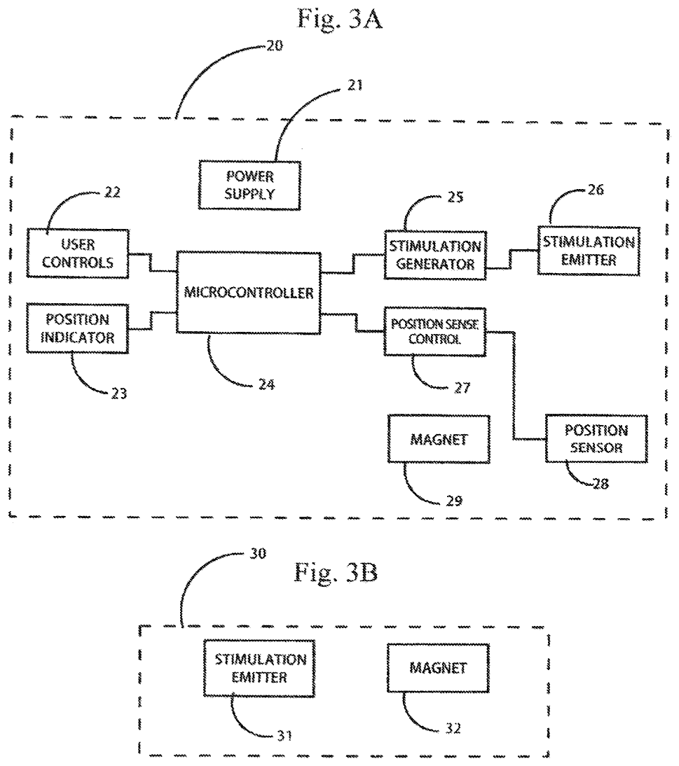

### REFERENCE NUMERALS IN DRAWINGS

1. Collar
2. Vagus Nerve Target
3. Superior Anti-Rotation Elastomeric Bristles
4. Inferior Anti-Rotation Elastomeric Bristles
5. Moveable Electrode car (position adjustable)
6. Superior (dorsal) Visual Position Indicator
7. Collar Pin ports
8. Enclosure Car Position—Locking pin port
9 Locking Pin—Spring Loaded?
10. Positive electrode contact post
11. Negative electrode contact post
12. Battery Pack+Stimulator electronics
20 Collar Mounted Enclosure Car
21 Battery, recharging and power supply electronics
22 User controls including power and mode select
23 Position indicator; visual and/or acoustic to indicate alignment with implanted capsule magnet; may be implemented by means of a Hall device.
24 Microcontroller
25 Stimulation generator; electronics controlled by microcontroller to set stimulation intensity
26 Stimulation emitter; may be of at least one of electrical, haptic or optical energy
27 Position sense control; electronics to condition output of position sensor.
28 Position sensor; magnetic sensor such as a Hall device to determine proximity of capsule magnet.
29 Magnet; collar; for magnet coupling of collar emitter with implanted capsule
30 Implanted stimulation capsule
31 Stimulation emitter; includes the inductive coupling antenna, current control electronic elements, and electrical contact or haptic element.
32 Magnet, capsule; for magnet force coupling alignment of capsule with collar magnet; to provide magnetic proximity for collar position sensor.

### DESCRIPTIONS OF PREFERRED AND SYSTEM EMBODIMENTS

### Various Embodiments Include:

1. Collar with local control, power supply and stimulation electronics directly driving at least one of:
a. Electrical emitters for direct contact applied to mammal subject's skin
b. Haptic emitters for application to mammal subject's skin.
   2. Collar with wireless control, local power supply and stimulation electronics directly driving at least one of:
a. Electrical emitters for direct contact with mammal subject's skin.
b. Haptic emitters for application to mammal subject's skin.
   3. Collar with local control and stimulation electronics inductively driving an implanted subcutaneous injected of at least one:
a. Capsule to generate electrical stimulation.
b. Capsule to generate haptic stimulation.
   4. A stimulation control system utilizing electromagnetic field transmitter to directly inductively power an implanted subcutaneous injected capsule without a collar. Said electromagnetic field may be transmitted by means of at least one of:
a. A floor mat for specified range application.
b. A room area antenna for wide range application.
c. A human worn glove or garment for close range application.

Said implanted capsule provides at least one of:
a. Electrical stimulation
b. Haptic stimulation

Said simulation signal waveforms including frequency and duty cycle may be modulated by said transmitter.

5. A stimulation control system utilizing electromagnetic field to inductively power collar worn stimulation electronics. Said electromagnetic field may be transmitted by means of at least one of:
a. A floor mat for specified range application.
b. A room area antenna for wide range application.
c. A human worn glove or garment for close range application.

We claim:

1. An improved energy neurostimulation system for stimulating at least one cranial nerve in the neck of a mammal includes an energy-generation package, energy stimulation means, controller means for controlling said energy stimulation means, and a neck worn coupling means for coupling at least one energy emitter circuit to at least one anatomical landmark corresponding with said at least one cranial nerve target, wherein:
said energy-generation package includes a power source, recharging electronics, and electronic controller for controlling and conditioning electrical energy;
said energy stimulation means includes a stimulator producing stimulation energy, stimulation control and conditioning electronics, having at least one channel of stimulation energy output;
said controller means communicates electronically with said energy-generation package and said energy stimulation means and includes at least one controller algorithm;
said neck worn coupling means is configured for coupling a said at least one energy emitter circuit to the neck of a said mammal;
said at least one energy emitter circuit communicates electronically with said energy stimulator means and said controlling means;
said at least one energy emitter circuit is selected from a group that includes emitters of electrical energy and emitters of haptic energy;
said at least one algorithm included in said controller means is configured to control at least one energy stimulation parameter selected from a group that includes power amplitude, fluence, waveforms, pulse widths, phase characteristics, stimulation frequencies, stimulation session periods, stimulation duty cycle, periodicity of stimulation, total energy delivered and the like;
said neck worn coupling means includes at least one position verification means for verifying the alignment of said at least one energy emitter circuit with a said at least one cranial nerve target;
said neck worn coupling means includes position stabilization means to retain the position of said at least one energy emitter circuit relative to the position of a said at least one cranial nerve target.

2. The energy neurostimulation system of claim 1, wherein:
said neck worn coupling means includes a flexible, elongated, collar apparatus configured to be worn by a said mammal;
said at least one energy emitter circuit is attached, in part or in whole, to said flexible, elongated, collar apparatus.

3. The energy neurostimulation system of claim 2, wherein:
said at least one energy emitter circuit is installed, in part or in whole, on at least one positionally adjustable car assembly attached to said flexible, elongated, collar apparatus;
said at least one adjustable car assembly includes position-locking means to secure its position on said flexible, elongated, collar apparatus;
said position stabilization means includes at least one elastomeric composition affixed to said flexible, elongated, collar apparatus;
said at least one elastomeric composition is composed to impede rotational movement of said flexible, elongated, collar apparatus around a said mammal's neck;
said flexible, elongated, collar apparatus includes said at least one position verification means configured as at least one visually observable marker.

4. The energy neurostimulation system of claim 3, further comprising at least one electronic controller device selected from a group that includes a conventional desktop computer, a portable notebook-type computer, a smartphone, a tablet, a dedicated electronic fob, and a wearable electronic control device, and the like;
said at least one electronic controller device includes hardware and software configured for wireless electronic communication with said controller means and said energy stimulation means;
said controller means includes energy monitoring means configured to monitor the connection between said at least one energy emitter means and said mammal's skin for at least one factor belonging to a group that includes electrical conductivity or electrical resistance;
said energy monitoring means are configured for monitoring energy emitted by said energy emitter means;
said controller means is configured to detect moisture proximal to electrical stimulation contacts on said mammal's body;
said controller means is configured to disable energy stimulation upon the detection of said moisture.

5. The energy neurostimulation system of claim 4, wherein:
said at least one cranial nerve target is the cervical branch of the vagus nerve.

6. The energy neurostimulation system of claim 5, further comprising a graphical user interface configured to provide selective control of at least two stimulation parameters belonging to a group that includes power amplitude, fluence, waveforms, wavelengths, pulse widths, phase characteristics, stimulation channels, stimulation frequencies, stimulation session periods, signal duty cycle, and time intervals of stimulation delivery, stimulation periodicity, total energy delivered, and the like.

7. The energy neurostimulation system of claim 6, wherein:
said at least one electronic controller device includes hardware configured to enable internet connectivity and the communicative exchange of data with at least one remote server;
said at least one computer device includes software configured for remote control of said energy stimulation by a remote operator via said internet connectivity.

8. The energy neurostimulation system of claim 7 wherein:
said at least one energy emitter circuit includes electrodes configured to emit electrical energy;
said electrodes emit electrical stimulation current having at least one frequency selected from a range of electrical frequencies between 0.5 hertz to 20,000 hertz;
said electrodes emit electrical stimulation in pulses having a pulse width between 100 and 1000 milliseconds;
said electrodes emit electrical stimulation in duty cycles having a stimulation-on periodicity between 10 seconds and 5 minutes;
said electrodes emit electrical stimulation in duty cycles having a stimulation-off periodicity between 10 seconds and 5 minutes.

9. The energy neurostimulation system of claim 7 wherein:
said at least one energy emitter circuit includes emitters of haptic, vibrational energy;
said emitters of haptic vibrational energy emit vibrational energy at frequencies in the range of 0.5 Hertz to 15,000 Hertz;
said emitters of haptic, vibrational energy emit haptic stimulation in pulses having a pulse width between 100 and 1000 milliseconds;
said emitters of haptic, vibrational energy emit haptic stimulation in duty cycles having a stimulation-on periodicity between 10 seconds and 5 minutes;
said emitters of haptic, vibrational energy emit haptic stimulation in duty cycles having a stimulation-off periodicity between 10 seconds and 5 minutes.

10. The energy neurostimulation system of claim 9, wherein:
said at least one energy-generation package includes a power source and electronic controller for controlling and conditioning electrical energy;
said at least one electromagnetic transducer is in electronic communication with said at least one energy-generation package;
said at least one electromagnetic transducer is configured to convert electricity into an electromagnetic field;
said at least one electromagnetic transducer is affixed to said neck worn coupling means;
said at least one receiver-emitter means includes an electromagnetic induction receiver and a haptic vibration-generation means;
said at least one receiver-emitter means is comprised to receive electromagnetic energy generated by said at least one transducer of electromagnetic energy and to produce haptic, vibrational energy;
said at least one electromagnetic transducer is installed on said at least one adjustable car attached to said flexible, elongated, collar apparatus;
said at least one adjustable car includes position-locking means to secure its position on said flexible, elongated, collar apparatus;
said neck worn coupling means includes at least one position verification means for verifying the alignment of said at least one electromagnetic transducer in relation to said at least one receiver-emitter means;
said neck worn coupling means includes position stabilization means to retain the position of said at least one electromagnetic transducer relative to the position of a said at least one receiver-emitter means;
said electromagnetic transducer and receiver-emitter means are configured to emit haptic vibrational energy at frequencies in the range of 1 Hertz to 15,000 Hertz.

11. The energy neurostimulation system of claim 10 furtherer wherein:
said at least one haptic vibration-generation means includes at least one magnetic component having a north pole and a south pole;
said at least one receiver-emitter means is comprised within a biocompatible package configured for percutaneous implantation subcutaneously under the skin of said mammal;
said at least one position verification means includes a Hall sensor configured to detect and monitor the proximity and position of a said at least one said magnetic component;
said Hall sensor includes signaling means to provide installation guidance and positioning monitoring to facilitate positioning and alignment of said at least one electromagnetic transducer proximal to said at least one haptic vibration-generation means.

12. The energy neurostimulation system of claim 11 wherein:
said biocompatible package containing said at least one receiver-emitter means is composed of glass, plastic or a combination thereof,
said Hall sensor detector means is used to signal from a group that includes a light source and a sound generator, each producing stimulus readily detectable by ordinary human sensory faculties;
said biocompatible package containing said at least one receiver-emitter means additionally includes a radio frequency identification (RFID) circuit.

13. The energy neurostimulation system of claim 8, further comprising at least one electrical output inductive transducer-transmitter, at least one receiver-emitter means, and a neck worn coupling means for coupling said at least one electromagnetic transducer to at least one anatomical landmark corresponding with said at least one cranial nerve target, wherein:
said at least one electromagnetic transducer is in electronic communication with said energy-generation package;
said at least one electromagnetic transducer is configured to convert electricity into an electromagnetic field;
said at least one electromagnetic transducer is installed on said adjustable car removably attached to said neck worn coupling means;
said at least one receiver-emitter means is comprised to receive electromagnetic energy generated by said at least one transducer of electromagnetic energy;
said at least one receiver-emitter means includes an electromagnetic induction receiver to convert electromagnetic energy into electric current for electrical stimulation of said at least one cranial nerve target;

14. The energy neurostimulation system of claim 13, wherein:
said at least one receiver-emitter means includes at least one elongated magnetic component having a north pole and a south pole;
said at least one receiver-emitter means is comprised within a biocompatible package configured for percutaneous implantation subcutaneously under the skin of mammal;
said at least one position verification means includes a Hall sensor configured to detect and monitor the proximity and position of a said at least one elongated magnetic composition included in said at least one receiver-emitter means;
said at least one elongated magnetic component has a north pole and a south pole;
said Hall sensor includes signaling means to provide installation guidance and positioning monitoring to facilitate positioning and alignment of said at least one electromagnetic transducer proximal to said at least one receiver-emitter means;

15. The energy neurostimulation system of claim 14, wherein:
said biocompatible package containing said at least one receiver-emitter means is composed of glass, plastic or a combination thereof,
said Hall sensor signaling means is selected from a group that includes a light source and a sound generator, each producing stimulus easily detectable by ordinary human sensory faculties;
said biocompatible package containing said at least one receiver-emitter means additionally includes a radio frequency identification (RFID) circuit.

16. The energy neurostimulation system according to claims 8, 9, 10, 11, 12, 13, 14, and 15 wherein:
said neck worn coupling means and controller means are configured for modular interchangeability of said emitters of electrical energy and said emitters of haptic energy;
said controller means is configured to electronically recognize and communicate with said modular said energy emitter means;
said controller is configured to control selected said energy emitter circuit according to established parameters associated with the said energy emitter circuit module selected for use.

17. The energy neurostimulation system according to claim 8, 9, 10, 11, 12, 13, 14, 15 and 16, further comprising:
at least one biofeedback sensor means configured for monitoring the cardiopulmonary activity of a said mammal;
said at least one biofeedback sensor means is configured to monitor at least one cardio-respiratory parameter belonging to a group that includes heart rate, respiratory rate, heart rate variability, arrhythmia, normal sinus rhythm, oxygen saturation and blood pressure;
said at least one controller algorithm is configured to control the delivery of energy stimulation in accordance with algorithm determinants and data obtained from said biofeedback sensor means.

18. The neurostimulation system according to claim 17, wherein:
said at least one biofeedback sensor means includes at least one photoplethysmography sensor configured to detect and monitor cardiologic activity of a mammal, particularly respiratory sinus arrhythmia, normal sinus rhythm and pathological heart rhythms also known as arrhythmias;
said detection of said respiratory sinus arrhythmia (RSA) is accomplished by monitoring to detect the periods of heart rate acceleration and deceleration associated with periods of said respiratory sinus arrhythmia (RSA);
said energy stimulation is gated according to said at least one algorithm configured to commence or end periods of energy stimulation in relation to the detection of said respiratory sinus arrhythmia;
said energy stimulation is gated according to said at least one control algorithm configured to commence or end periods of energy stimulation in relation to the detection of said normal sinus rhythm;
said monitoring of said cardiologic activity of a said mammal may be used to adjust said at least one stimulation parameter belonging to a group that includes stimulation frequency, waveform, pulse rate, pulse width, stimulation amplitude (i.e., intensity), stimulation duration, stimulation periodicity, and the like;
said monitoring of said cardiologic activity of a said mammal may be used to commence, delay or terminate stimulation according to detected pathological heart rhythms known as arrhythmias.

19. The neurostimulation system according to claim 19, wherein:
at least one bodily activity sensor means is an actimetry sensor configured for sensing and monitoring the bodily movement of a said mammal;
said at least one controller algorithm is configured to control the delivery of energy stimulation in accordance with algorithm determinants and data obtained from said bodily activity sensing means.

20. The neurostimulation system according to claim 19 further comprised as an algorithm-operated and algorithm-controlled closed-loop system, wherein:
said photoplethysmography is further configured to continuously monitor at least one index of autonomic nervous system activity;
said at least one index of photoplethysmographically monitored autonomic nervous system activity includes one or more frequency domains of Heart Rate Variability selected from a group that includes high frequency, low frequency, very low frequency and ratios thereof, and the like;
said bodily activity sensors further comprise sensors configured for monitoring the acceleration, motion, and position of the body in whole or in part;
said at least one algorithm is composed for selectively controlling one or more stimulation parameters according to algorithm determinants and said at least one index of photoplethysmographically monitored autonomic nervous system activity, and data from said biofeedback sensors and bodily movement sensors;
said closed loop system may be comprised as a self-contained wearable system having a power source, and energy conditioning control electronics;
said one or more stimulation parameters controlled by said algorithm are selected from a group that includes energy frequency, energy intensity, stimulation time duration, energy pulse width, energy waveform, stimulation duty cycle, power amplitude, fluence, waveforms, wavelengths, pulse widths, phase characteristics, stimulation channels, stimulation session periods, signal duty cycle, periodicity of stimulation, total energy delivered and the like.
